(19) United States
(12) Patent Application Publication
(10) Pub. No.: US 2003/0130645 A1
(43) Pub. Date: Jul. 10, 2003

(54) Devices and methods for delivery of medically appropriate fluids

(75) Inventors: David R. Brengle, San Diego, CA (US); Marc S. Lieberman, Carlsbad, CA (US); Mark C. Doyle, San Diego, CA (US); Daniel Z. Glazerman, San Diego, CA (US); Jeremy D. Fennelly, Escondido, CA (US)

Correspondence Address: Michael A. Whittaker, Foley & Lardner, P.O. Box 80278, San Diego, CA, 92138-0278, US

(73) Assignee: Tandem Medical, Inc.

(21) Appl. No.: 10/251,941

(22) Filed: Sep. 19, 2002

Related U.S. Application Data
(60) Provisional application No. 60/337,407, filed on Dec. 3, 2001.

(51) Int. Cl. A61M 31/00

(52) U.S. Cl. 604/500; 604/151

(57) ABSTRACT

The present invention relates to delivery containers designed to deliver fluids for infusion to patients in a predetermined sequence, and methods for their construction and use. The containers described herein integrally comprise a plurality of non-fluidly connected chambers. The containers may be configured to deliver a volume of each medication of an infusion therapy in a predetermined sequence, duration, and/or interval from these chambers; alternatively, a container may be part of a larger device that provides the necessary hardware to perform such predetermined delivery. The container provides improved infusion therapy administration by reducing opportunities for error, infection, adverse drug interactions, or other complications.

This application claims priority to U.S. Provisional Patent Application No. 60/337,407, filed Dec. 3, 2001 (abandoned); to U.S. patent application Ser. No. 09/713,521, filed Nov. 14, 2000 (pending), which is a divisional of U.S. patent application Ser. No. 09/231,535, filed Jan. 14, 1999, which issued as U.S. Pat. No. 6,146,360, which is a continuation of U.S. patent Ser. No. 09/008,111, filed Jan. 16, 1998, which issued as U.S. Pat. No. 6,074,366; to U.S. patent application Ser. No. 09/434,972, filed Nov. 5, 1999 (pending); and to U.S. patent application Ser. No. 09/434,975, filed Nov. 5, 1999, each of which is hereby incorporated by reference in their entirety, including all tables, figures, and claims.

### FIELD OF THE INVENTION

The present invention generally relates to devices and methods for the delivery of medication and/or other fluids in accordance with a predetermined medical therapy.

### BACKGROUND OF THE INVENTION

Intravenous medications including antibiotics and the like may be administered intermittently over an extended period of time. Each administration of an intravenous therapy generally follows a predefined procedure that often includes a series of manual steps. Such manual steps may include saline flushes and generally terminate with the application of anti-clotting medication. The manual steps in the therapy procedures are a principle source of error, infection, and other complications that may arise during intermittent infusion therapy.

Examples of medication delivery containers and medication delivery pumps have been described in U.S. Pat. No. 6,146,360; U.S. Pat. No. 6,074,366; U.S. patent application Ser. No. 09/434,972, filed on Nov. 5, 1999; and U.S. patent application Ser. No. 09/434,974, filed on Nov. 5, 1999; each of which is hereby incorporated by reference in its entirety, including all tables, figures, and claims.

There remains a need in the art for a devices and methods to improve the administration of intermittent medication infusion therapy. The present invention satisfies this and other needs in the art.

### BRIEF DESCRIPTION OF THE INVENTION

The present invention describes medication delivery containers designed to deliver fluids in a predetermined sequence, and methods for their construction and use. The containers described herein comprise a plurality of non-fluidly connected chambers that are integral to the container. The phrase “integral container” is defined hereinafter. The integral containers of the present invention may be configured to deliver a volume of each fluid in a selected infusion regimen in a predetermined sequence, duration, volume, and/or interval from these chambers. Alternatively, a container may be part of a larger device that provides additional hardware to perform the desired sequential delivery. The container provides improved infusion therapy administration by reducing opportunities for error, infection, adverse drug interactions, or other complications.

In various embodiments, fluids may be delivered from the integral containers of the present invention by application of positive pressure to one or more non-fluidly connected chambers, negative pressure to one or more non-fluidly connected chambers, by gravity feed from one or more non-fluidly connected chambers, or by some combination of these delivery modes.

In certain preferred embodiments, positive pressure is created by compression of a chamber within the integral containers of the present invention, thus expressing fluid from that chamber through a port in the chamber wall. In these embodiments, the chamber is preferably flexible, and positive pressure may be generated in a plurality of chambers in a predetermined sequence, for example, by a roller pump compressing each chamber at the proper time, thereby delivering the fluids from the integral container in the desired sequence. Other means of generating positive pressure, such as injection of a gas or other fluid into a chamber to express some or all of the contents of that chamber, are also contemplated by the present invention

In other preferred embodiments, negative pressure is created by application of a pump to an output port or conduit fluidly connected to a chamber within the integral containers of the present invention, thereby extracting fluid from that chamber through a port in the chamber wall.

Controlled fluid flow from the integral containers of the present invention may be obtained using a variety of methodologies. For example, force (either positive, negative, or gravity) may be used to deliver fluid from one or more chambers within the intergral container in sequence. This may be achieved, e.g., by allowing a single pump to access a plurality of chambers in sequence; or by having multiple pumps, each of which may be connected to a corresponding chamber, and actuating the pumps in sequence. Alternatively, valves that control flow from each chamber may be actuated (manually, electronically, pneumatically, etc.) in sequence, thereby permitting flow to occur from a given chamber. The skilled artisan will understand that such control means need not be selected individually, and that a given device might include control at both the pump and valve level for example.

In certain preferred embodiments, fluid flows from a plurality of chambers to a manifold that receives flow from several input conduits, and that generates flow through a single exit conduit. The functionality of a manifold may also be served by other flow structures, such as a set of multi-path (e.g., three-way) valves or connections placed in series. In such embodiments, each multi-path connection might receive flow from a previous chamber or valve, as well as from a new chamber, with a resulting flow to a single exit conduit or port.

In preferred embodiments, the integral containers of the present invention may be constructed as a flexible bag having a plurality of non-fluidly connected chambers. Such containers may also include structures for minimizing pressure drop which may be associated with a chamber upon the application of pressure to the respective chamber, thereby allowing relatively unimpeded fluid flow from the respective chamber to an associated conduit during the application of pressure to the chamber.

While the present invention relates in part to containers that may be provided to a medical provider (e.g., physician or pharmacist) or other user in an unfilled state for subsequent filling in a manner deemed appropriate by that user, in various aspects the invention also relates to containers in which one or more, and preferably all, chambers within the container are provided to a user in pre-filled with fluids to be delivered in a predetermined sequence.

The foregoing summary of the invention is non-limiting, and other features of the invention will be apparent to those of skill in the art from the following figures, detailed description of the invention, and the claims.

### BRIEF DESCRIPTION OF THE FIGURES

FIG. 1 is a schematic view of an integral container according to the invention, showing a plurality of non-fluidly connected chambers connected to a set of valves in series (A), in parallel (B), and in combination of the two (C).

FIG. 2 is a schematic view of an integral container according to the invention, showing a plurality of non-fluidly connected chambers connected to a set of valves in parallel. The figure shows two possible locations for valves (i.e., within the integral container itself, or within an external flow path leading from each chamber), together with possible locations for an accumulator chamber.

FIG. 3 is a schematic view of an integral container according to the invention, showing a plurality of non-fluidly connected chambers connected to a set of actively-controlled valves and single pump to generate flow using negative pressure.

FIGS. 4 and 5 collectively show an exemplary pumping sequence, using a shuttle valve to provide sequential delivery from each chamber in an integral fluid delivery container.

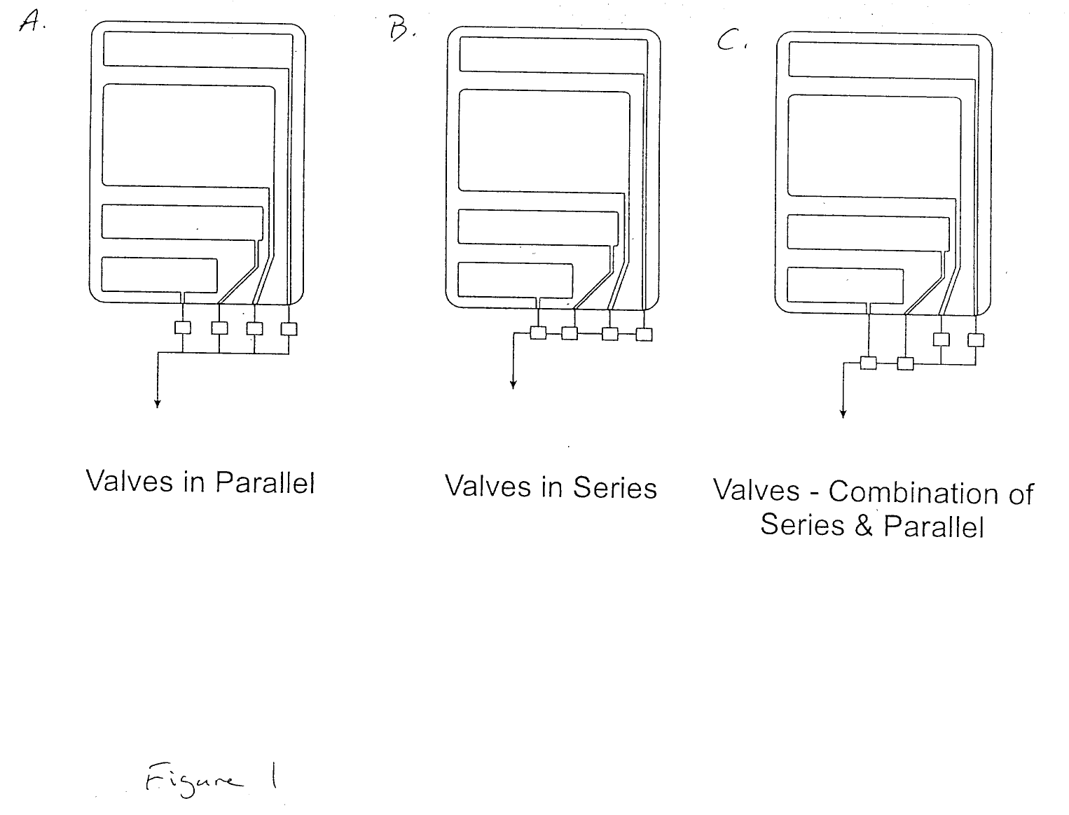

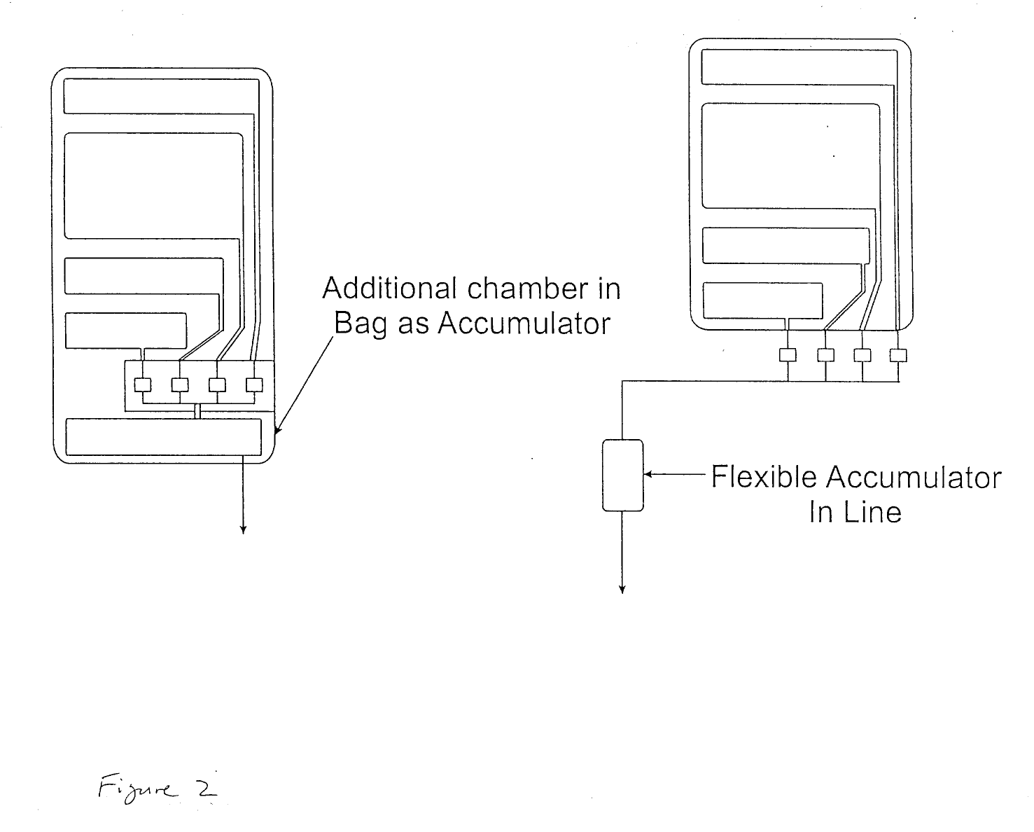

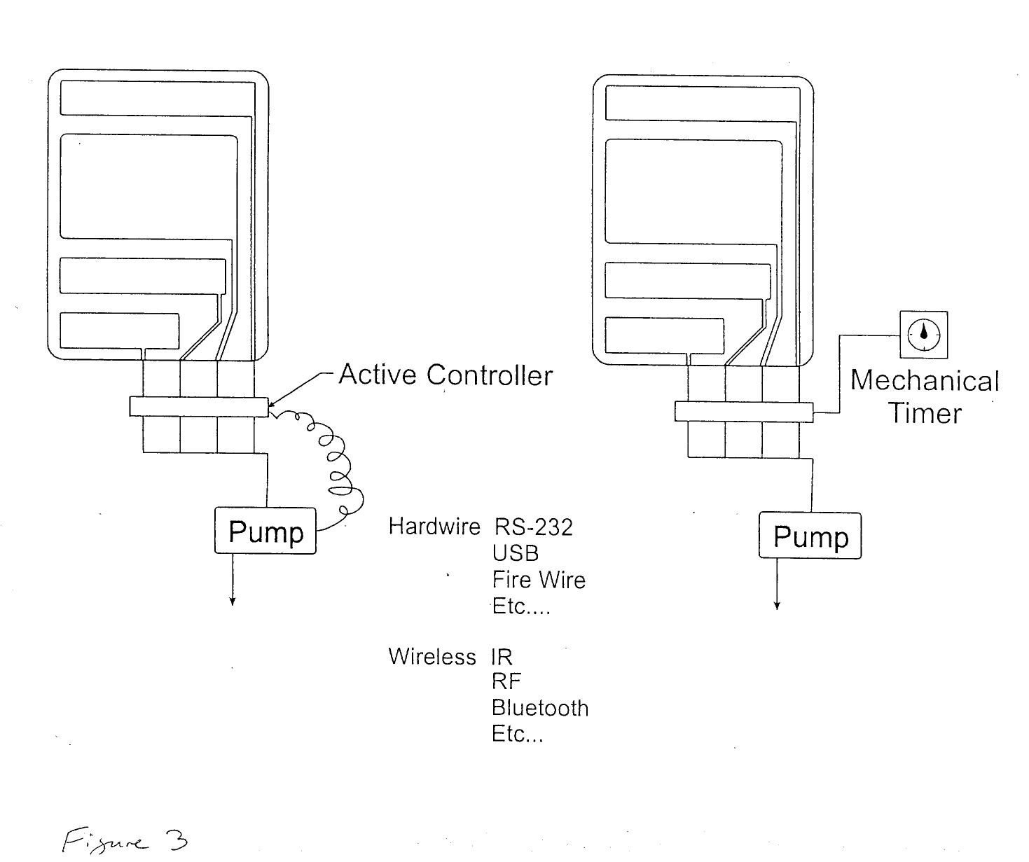

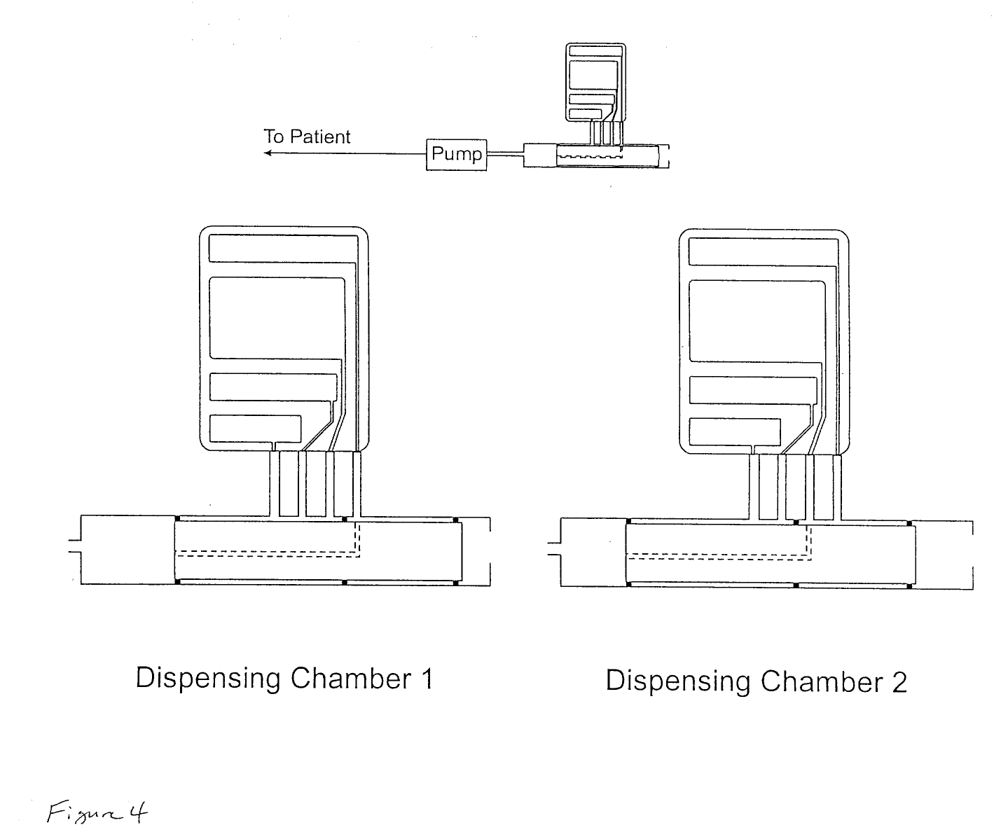

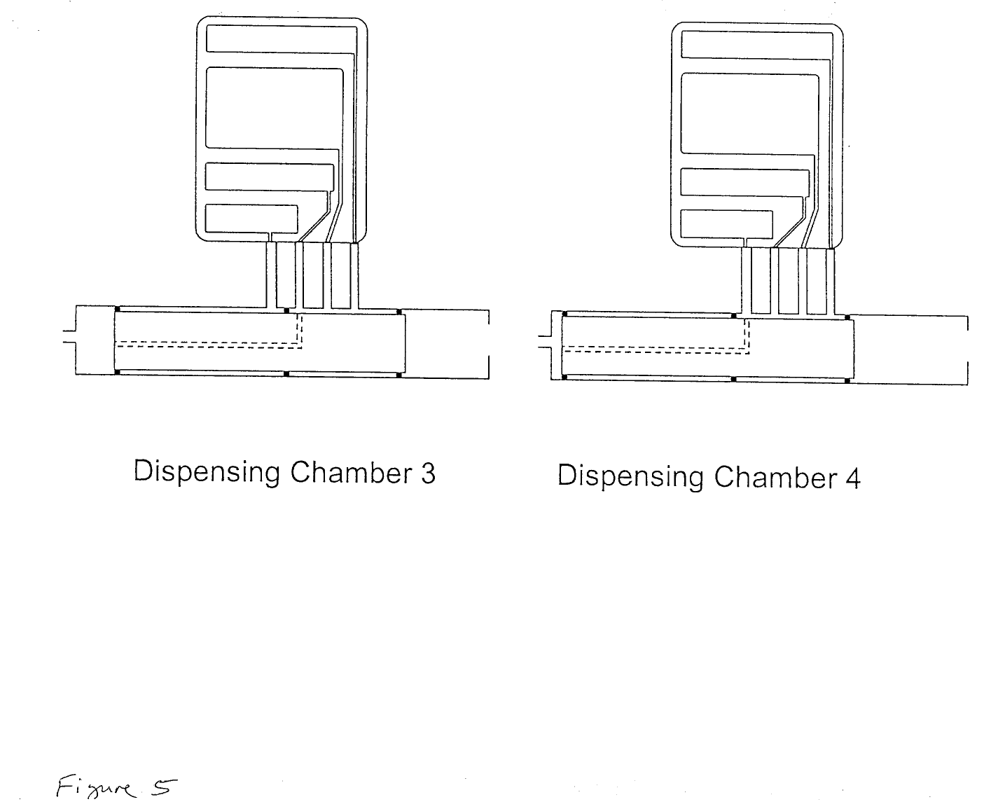

### DETAILED DESCRIPTION OF THE INVENTION

In accordance with the present invention, there are provided medication delivery containers designed to deliver fluids in a predetermined sequence, and methods for the construction and use thereof. The containers described herein comprise a plurality of non-fluidly connected chambers that are integral to the container. The containers may be configured to deliver a volume of each fluid of an infusion therapy regimen in a predetermined sequence, duration, volume and/or interval from these chambers; alternatively, a container may be part of a larger device that provides the necessary hardware to perform such sequential delivery.

Fluids may be delivered from the non-fluidly connected chambers by gravity, by the generation of positive pressure within a chamber, by the generation of negative pressure within a chamber, or by a combination of the above. Control of this fluid flow may be obtained by careful configuration of the geometry of the chambers and conduits within the container, by controlled pump actuation, by controlled valve actuation, or by a combination of such control means.

The phrase “integral container” as used herein refers to a container comprising a plurality of non-fluidly connected chambers, in which removal of a chamber would result in a loss of integrity of the entire container. For example, a preferred embodiment of the integral containers of the present invention is a flexible bag in which the chamber walls are formed from the container walls. Thus, in these embodiments, removal of a chamber would also entail removal of a portion of the container itself. By way of contrast, U.S. Pat. No. 5,658,271 discloses a device in which individual containers are placed in a housing. In this non-integral container, each bag may be replaced without disrupting the integrity of the larger housing.

The phrase “non-fluidly connected” as used herein in reference to chambers within an integral container refers to an absence of fluid connections between the chambers themselves that would allow fluids to intermingle before flowing from one of the chambers to a patient. Such chambers may intermingle fluids at a point downstream from the chambers, such as at a manifold, but the chambers from which the fluids originate would still be non-fluidly connected. Additionally, such chambers may be connected, such as via a conduit, but so long as no fluids to be delivered from each chamber to a patient intermingle before their delivery out of the chambers, the chambers would still be said to be non-fluidly connected.

The phrase “substantially non-fluidly connected” as used herein in reference to chambers within an integral container refers to chambers in which fluid connections between the chambers allow less than 10% of fluids from one chamber to intermingle with the fluids in another chamber before flowing from one of the chambers to a patient. More preferably, chambers that are substantially non-fluidly connected allow less than 5%, and most preferably less than 1%, of fluids from one chamber to intermingle with the fluids in another chamber before flowing from one of the chambers to a patient.

The phrase “fluidly connected” as used herein in reference to chambers within an integral container refers to chambers in which fluid connections between the chambers allow 10% or more of the fluids from one chamber to intermingle with the fluids in another chamber before flowing from one of the chambers to a patient. Such fluidly connected chambers may originate as non-fluidly connected or substantially non-fluidly connected chambers, but be rendered fluidly connected prior to delivery of fluids from one of the chambers. For example, a frangible seal between chambers may be breached, allowing the fluids in the chambers to intermingle. Preferably, fluidly connected chambers allow 50% or more, and most preferably 90% or more, of the fluids from one chamber to intermingle with the fluids in another chamber before flowing from one of the chambers to a patient.

The phrase “predetermined sequence” refers to delivery of a plurality of fluids from an integral container to a patient according to a treatment regimen desired by a clinician. Such a predetermined sequence may involve delivery of fluids discretely, i.e., a first fluid is completely delivered before a second fluid is delivered, or in an overlapping manner, i.e. all or a portion of a second fluid is delivered at the same time that a first fluid is delivered. The predetermined sequence may include both controlled timing of delivery, volume of delivery, and/or order of delivery.

The phrase “positive pressure” as used herein refers to the application of force to a fluid or chamber resulting in fluid pressure within a chamber that is greater than the force of gravity; that is, a pressure greater than that created by the hydrostatic head pressure within the chamber. Such positive pressures may be generated by a pump or other means of pushing on a fluid or chamber. Suitable positive pressures are any pressure that the chamber may withstand without breaching the integrity of the chamber (e.g., bursting). Preferred pressures are between 100 psi and 0.1 psi, more preferably between 40 psi and 0.5 psi, and most preferably between 10 psi and 1 psi.

Similarly, the phrase “negative pressure” refers to the application of force to a chamber resulting in fluid pressure within a chamber or conduit that is less than the force of gravity. Such pressures are often referred to as “suction pressures” or “vacuum pressures.” Negative pressures may be generated by a pump or other means of pulling fluid from chamber. Suitable negative pressures are any pressure that the chamber, or any conduit between the chamber and the source of the negative pressure, may withstand without collapsing. Preferred pressures are between 5 psi and 0.1 psi, more preferably between 3 psi and 0.25 psi, and most preferably between 1 psi and 0.5 psi.

The phrase “gravity feed” refers to the use of only gravitational forces to deliver a fluid. The skilled artisan will understand that gravity feed methods may be used to differentially deliver fluids from two or more chambers, e.g., by varying the head height of each chamber.

The term “manifold” as used herein refers to a discrete structure that receives fluid flow from a plurality of input ports, and allows a resulting flow through a reduced number of output ports. In preferred embodiments, a manifold receives flow from at least three input ports, and allows a resulting flow through a single output port. In particularly preferred embodiments, a manifold receives flow from each non-fluidly connected chamber through a corresponding number of input ports, and allows a resulting flow through a single output port. Flow from one or more input ports to an output port in a manifold may be passive, or may be controlled by one or more valves.

The term “upstream” as used herein refers to any point in a flow path that is closer to the source of the flow path than to the destination of the flow path. An upstream point may also be referred to as a “proximal” location. Similarly, “downstream” refers to any point in a flow path that is closer to the destination of the flow path than to the source of the flow path. A downstream point may also be referred to as a “distal” location.

The phrase “control device” as used herein refers to any device that can reversibly modulate flow down a particular flow path. Control devices of the present invention may be active or passive, as described below, or may serve both an active or passive control function.

The term “valve” as used herein refers to any device within a flow path that starts, stops, or modulates flow through the flow path. Suitable valve configurations are well known to those of skill in the art, including umbrella valves, disc valves, poppet valves, duckbill valves, ball valves, and flapper valves, shuttle valves, gate valves, slit membrane, check valves, and the like.

The phrase “active control device” as used herein refers to any device that can reversibly modulate flow down a particular flow path, and which is not actuated by only the flow or pressure within the flow path itself. An active control device can be one intended to be operated manually, such as a manual valve, stopcock or a pinch clamp, or can be a valve or stopcock that is operated pneumatically, hydraulically, mechanically, by vacuum, or electrically for example. Active control devices may be located within the integral container itself, or along a flow path (e.g., within or along a conduit) between a chamber and the patient. In preferred embodiments, an active control device can reversibly halt all flow down a particular flow path.

The phrase “passive control device” as used herein refers to any device that can reversibly modulate flow down a particular flow path, and which is actuated by only the flow or pressure within the flow path itself. A passive control device can be a valve or stopcock that is opened or closed by altering the flow rate or pressure at the passive control device location. Passive control devices may also be located within the integral container itself, or along a flow path (e.g., within or along a conduit) between a chamber and the patient. In preferred embodiments, a passive control device can reversibly halt all flow down a particular flow path.

As discussed herein, an integral container can be formed from any material from which a container comprising a plurality of integral, non-fluidly connected chambers may be fabricated. As will be appreciated by those of skill in the art, any suitable biocompatible material may be employed in the construction of the integral container, however, it is presently preferred that at least one side of the integral container be transparent to facilitate viewing of the contents.

While the skilled artisan will understand that an integral container may be formed from a variety of material configurations, it is presently preferred that the container be formed of two sheets of flexible material (athough three, four, or more sheets may also be used). For example, the flexible sheets may be ethyl vinyl acetate (EVA), polyvinyl chloride (PVC), polyolefin, or other suitable material: In one embodiment, the first sheet of flexible material has a relatively smooth inner surface and the second sheet of plastic has a texture, such as a taffeta texture (e.g., a diamond taffeta), ribs, or the like, embossed on its inner surface. Alternatively, both sheets may have a patterned inner surface, e.g., a raised diamond taffeta.. The sheets are joined together around the perimeter of the container by any means suitable for forming an air and fluid-tight seal that can withstand the pressure generated by the pump apparatus. Fluid-tight seals are also formed between the individual chambers, and should have the same minimum pressure tolerances as the perimeter seals. Thus, the sheets are bonded together to create the patterns for the chambers, conduits, and ports. The materials may be bonded in a variety of ways, e.g., by a radio frequency (rf) seal, a sonication seal, a heat seal, adhesive, or the like, to form an air and fluid-tight seal as described herein.

Each chamber of the integral container preferably has one or more associated conduits. The conduits provide a pathway for fluid to enter and/or exit each chamber. The conduits can be integrally formed during construction of the container, for example, by leaving channels unbonded when the two sheets are fused together to form the container. Optionally, additional internal structure (e.g., rigid or semi-rigid tubing, or the like) may be provided to facilitate fluid flow to and from each chamber.

In an integral container in which fluid is to be delivered by compression of one or more chambers, it is preferred that the conduit through which fluid exits such a chamber lies outside of the compression region (i.e., the region to which pressure is directly applied by contact with a pressure applying structure in the pump apparatus). In this manner, mixing of residual medications in the conduits with subsequently administered medications from other chambers can be minimized. Alternatively, the conduits may lie within the compression region, particularly if mixing is not a concern.

If the conduits are constructed by leaving unbonded channels in the integral container, the conduit will have a generally flat shape but enlarges to have a more tubular shape upon the application of pressure to the corresponding chamber. The shape of the conduit depends on the strength of the materials used to construct the integral container and the pressure of the fluid therein. Specifically, less flexible material may require greater pressure for enlarging the conduit. Advantageously, the textured inner surface of at least one side of the integral container provides flow channels that allow liquid pressure to act along the length of the conduit to assist in opening the conduit upon the application of pressure to the respective chamber. Otherwise, if both inner sides of the container are smooth, surface tension may hold them together and a greater amount of pressure may be required to open the conduits and initiate flow.

The skilled artisan will also understand that a variety of methods may be provided to provide sequential flow control from the various non-fluidly connected chambers within an integral container. One method of providing such control is to configure the integral container itself to provide such sequential flow. Methods and compositions for providing such integral containers are described in U.S. Pat. No. 6,146,360; U.S. Pat. No. 6,074,366; U.S. patent application Ser. No. 09/713,521; U.S. patent application Ser. No. 09/434,972; and U.S. patent application Ser. No. 09/434,974, each of which is incorporated by reference herein in its entirety, including all tables, figures, and claims.

In one embodiment of the present invention, the chambers and corresponding conduits from each chamber are arranged in the integral container so that when positive pressure is applied sequentially from one end of the integral container to the opposite end, individual chambers are sequentially activated. It is presently preferred that the pressure be applied evenly. Even, sequential application of pressure can be accomplished by employing a constant force spring, a roller attached to a constant force spring, a motor-driven roller, or the like.

Additionally, sequential flow control from the various non-fluidly connected chambers within an integral container can be provided by inclusion of one or more pumps, or other means for generating positive or negative pressures, along one or more flow paths between the integral container and the patient. For example, in embodiments where positive pressure is generated, each chamber may be connected to an independently controllable source of pressurization, such as a compressed gas source or a pressurization pump. Similarly, an independently controllable source of negative pressure (e.g., individual pumps or one or more multichannel pumps) can be placed along the flow path from the non-fluidly connected chambers. Suitable pumps are well known in the art. See, e.g., U.S. patent application Ser. No. 09/434,974; and U.S. Pat. Nos. 6,270,478; 6,213,738; 5,743,878; 5,665,070; 5,522,798; and 5,171,301, each of which is hereby incorporated by reference in its entirety, including all tables, figures, and claims. Pumps useful in the present invention can be simple pumps which are either “on” or “off,” or may comprise a programmable controller (referred to herein as a “smart pump”) that may be integral to the pump or exist as a separate controller unit interfaced in a wired (e.g., via hard wiring, a serial port (such as a standard RS-232 port), a USB port, a “fire wire” port, etc.) or wireless fashion (e.g., connected an via infrared connection, a radio frequency connection, a “bluetooth” connection, etc.).

Suitable programmable pumps are available that permit the operator to generate a pre-defined or user-defined pumping profile. Such pumps may be used to define a volume and rate for fluid flow from each chamber in the integral container. For example, in an integral container having 4 chambers of 10 mL, 100 mL, 10 mL, and 5 mL, the pump could be programmed to run at 1000 ml/hr for the volume of chamber 1, then 200 ml/hr for the volume of chamber 2, then 1000 ml/hr for the volume of Chamber 3, and finally 1000 ml/hr for the volume of chamber 4. Alternatively, four separate pumps (or the individual pumping heads of a 4-channel pump) could be individually or collectively programmed to perform this profile.

Similarly, a pump could be configured to determine a suitable rate, limited by a maximum rate threshold and maximum pressure threshold. In this embodiment, the pump would ramp up the pumping rate until some pre-set maximum rate or pressure was reached. Alternatively, a pump could deliver a “pulsatile” rate, alternating between a preset minimum and a preset or pump-determined maximum rate. These examples are not limiting, and additional pumping profiles could be readily determined by the skilled artisan.

As an alternative to, or in conjunction with one or more pumps for providing sequential flow, one or more active control devices can also be located along one or more flow paths between the integral container and the patient. In these embodiments, controlled actuation of the active control device(s) can provide the required flow control of fluids from the integral container. An active control device can be as simple as a manual pinch valve, which the operator will open as required by the sequential delivery method, or can be a more complicated electrically or pneumatically operated valve. In the latter case, the active control device can be integrally controlled, or can be connected to a controller unit in a wired fashion (e.g., via hard wiring, a serial port (such as a standard RS-232 port), a USB port, a “fire wire” port, etc.) or in a wireless fashion (e.g., connected an via infrared connection, a radio frequency connection, a “bluetooth” connection, etc.).

The various flow paths (ports, conduits, etc.) from each non-fluidly connected chamber to the patient will preferably merge at some point into a single conduit through which fluids are infused to the patient. Numerous methods are well known to the skilled artisan to merge such flow paths. These can include simple connections, such as 3-way (or 4-way, or 5-way, etc.) connectors in which two (or three, or four, etc.) input paths flow out through a single output path. In more complex arrangements, the placement of valves (arranged in parallel or series, or a combination of the two) on each flow path, and/or one or more manifold units can provide the required merger of flow paths. Exemplary manifolds are described hereinafter. Other manifolds are disclosed in, e.g., U.S. Pat. Nos. 5,374,248; 5,217,432; and 5,431,185, each of which is hereby incorporated by reference in its entirety, including all tables, figures, and claims. Manifolds may additionally contain one or more control devices, either active, passive, or a combination thereof, to control the flow of fluid through the manifold.

In embodiments where negative pressure is used to withdraw fluid from the chambers of an integral container, the skilled artisan will understand that the overall configuration of the device may depend on the position of the pump relative to the merger of the various flow paths. For example, a plurality of pumps (or a multichannel pump) corresponding to a plurality of flow paths flowing into a manifold may be placed upstream from the manifold, thereby providing a means to provide negative pressure along each flow path. Alternatively, a single pump placed downstream of a manifold may be used to provide negative pressure along each flow path that flows into the manifold.

It may be desirable to include an accumulator chamber, either in the integral container itself or on one or more flow paths leading from the container. Some positive displacement pumps have a relatively high flow rate when the displacement chamber in the pump is being refilled. If the refill flow rate is faster than the gravity flow rate from the multi-chambered container, fluid could potentially be pulled out of more than one chamber. To avoid this, an accumulator chamber that is preferably flexible, is placed in series with the pump. Fluid may be permitted to flow (e.g., by gravity) into the accumulator until it is full, and when the downstream pump pulls fluid, it will pull only from the accumulator. Flow out of the appropriate chamber will then re-fill the accumulator for the next fill stroke of the down stream pump.

It may also be desirable to mix the contents of two or more chambers immediately prior to administration to form a single chamber that is not fluidly connected to other chambers within the integral container. Accordingly, in another embodiment of the present invention, frangible seals between two or more adjacent chambers may be formed. The chambers may be side by side, parallel or perpendicular relative to one axis of the integral container.

Chambers may also be configured to have a “blow down” period between activation of one chamber and activation of the next chamber during an infusion sequence to prevent mixing of medications during the infusion. As described in greater detail below, this can be accomplished, for example, by providing a space between adjacent chambers, or the like.

In those embodiments where the integral container is of a flexible configuration (e.g., a bag) it has been observed that there can be a pressure drop between a chamber and its corresponding conduit when pressure is applied to the contents of the integral container. This is largely due to the formation of kinks in the flexible material when pressure is applied to the contents of the integral container. The region of primary concern is the interface between the chamber and its corresponding conduit. Thus in one embodiment of the present invention, structure is provided to alleviate pressure drop between each chamber and its corresponding conduit. This can be achieved by one or more of several methods, including quilting of the chamber, incorporation into the chamber of internal structures (e.g., a stent, tubing, conduit bead(s), solid filament, or the like), employing external structures (e.g., a source of pressure on the container, such as a protruding member of the pump apparatus, or the like), and the like. Additionally, the width, angle and/or taper of the conduit, the thickness of the chamber or conduit, and/or the type of material forming the chamber or conduit may be selected to minimize flow resistance.

As used herein, “quilting” means forming a structure in the interior of the chamber wherein the bottom and top sides of the integral container are connected, preferably by fusing them together. It is presently preferred that quilting be employed to manage pressure drop, as the desired connection between first and second sides of the integral container can be accomplished by the same methods used to form the perimeter seal of the container. Quilting may be at any region of the chamber that provides a substantially reduced or eliminated pressure drop between the chamber and its corresponding conduit. It is presently preferred that the quilting be in the region of the chamber that is proximal to the conduit. Suitable quilting configurations are described in U.S. patent application Ser. No. 09/713,521.

Other features suitable for minimizing flow resistance (i.e., pressure drop) caused by kinks include thermoforming of the conduit, introduction of an internal conduit bead in the region where the conduit joins the chamber, coining, or the like. Thermoforming involves heating the integral container materials in the region of the exit and associated conduit until the materials are softened slightly. Air pressure is applied to the chamber to open (or inflate) the exit and the conduit. The material is allowed to cool such that the exit and conduit retain a slightly circular opening or cross-section after the pressure is removed. In certain embodiments, a mold may be used to constrain the shape of the blow-molded conduit. For employing internal conduit bead(s), a portion of the bag adjacent the exit to the conduit is stamped with an offset bonding pattern or shim to provide a three-dimensional structure in the region of the exit. This can be analogized to gluing two sheets of paper together at their perimeter and affixing a solid piece, like a bamboo skewer along the length of the seam between the two sheets. In this manner, even when the two sheets are pressed together, a channel will exist along the skewer where the sheets are prevented from meeting one another. Additionally, coining (i.e., forming a structured pattern in the integral container material) may be applied to the sides of the integral container in the region of the exit to provide additional flow pathways not subject to greatly restricted flow by kinks.

It is contemplated that each conduit will have an associated port where, at a minimum, fluids exit the integral container. These conduits may serve the dual purpose of providing a channel for both the introduction of fluids into the chamber(s) and exit of fluids from the chambers. The container may have one or more ports for introduction of fluids into one or more of the individual chambers of the container. In one embodiment, these ports have associated conduits, separate from the exit conduits. The ports are configured to allow regulated, sterile introduction of fluids. This can be accomplished by fitting the ports with injection ports, or the like.

As discussed above, the fluid delivery devices of the present invention can comprise a manifold to regulate delivery of fluids from the ports on the integral container corresponding to each chamber to an administration tube set (“administration set”). Such a manifold may optionally provide a structure for filling one or more chambers. As used herein, “container port of the conduit” and “container port” refer to the terminal portion of each conduit leading to/from a chamber in the integral container. The container ports may have an adapter affixed thereto for mating the ports with the manifold, or the manifold may be attached directly to the container ports.

In describing the manifold, reference will be made to the “integral container side” of the manifold (where the manifold attaches to the integral container ports) and the “infusion side” (where the manifold attaches to the administration set). Further reference will be made to chamber ports of the manifold, where the manifold attaches to and is in fluid communication with the chamber ports. Additional reference will be made to an output port of the manifold, where the manifold attaches to and is in fluid communication with the administration set. Although optional, it is presently preferred that the manifold also have a bulk fill port, where the manifold can be attached to, and be in fluid communication with, a source of fluids for introduction into the integral container.

Manifolds contemplated for use in the practice of the present invention will have manifold conduits for directing fluid from chamber ports to the output port for exit to the administration set, and from the bulk fill port, when employed, to the chamber ports. These manifold conduits can be isolated from one another in a fluid-tight manner and can comprise internal chambers connecting the desired portions of the manifold, or they may comprise internally mounted tubing connecting the appropriate portions of the manifold, combinations thereof, or the like.

In order to regulate the flow of fluid through the manifold and to prevent backflow from the output port to the chamber ports, it is presently preferred that the manifold have check valves therein. Check valves can be configured in a variety of manners to regulate fluid flow as desired; all such configurations are contemplated as being within the scope of the present invention. In one embodiment of the present invention, fluid flow is regulated so that fluid exiting the container and entering the manifold through the chamber ports can only exit the manifold through the output port without returning to the bag by way of any other chamber port. This is accomplished by interposing a first check valve in a first conduit between each chamber port and the output port. The check valve only allows fluid to flow from the bag side of the manifold towards the infusion side where the output port is located.

It is important to note that some or all of the chambers may be individually filled by way of optional separate fill ports on the integral container and/or by way of the optional bulk fill port of the manifold. In an embodiment of the present invention, when a bulk fill port is to be used, fluid flow in the manifold is further regulated so that fluid introduced through the bulk fill port can access one or more of the chamber ports for filling of chambers in the integral container. Accordingly, chamber ports to be used for both filling and dispensing fluids will have two manifold conduits associated therewith: a first manifold conduit, as described above, for directing fluids from the chamber port(s) to the output port; and a second manifold conduit branching off of the first at a point between each chamber port and the first check valve. In this embodiment, a second check valve is located on each second manifold conduit between the chamber port and the bulk fill port. The second check valve only allows fluid to flow from the bulk fill port towards the chamber port.

The ports, valves and conduits of the manifold may be configured in any manner that permits the desired flow of fluid through the manifold. It is presently preferred that the conduits and output port be configured so that fluid exiting each sequentially activated bag chamber flows through its associated first check valve and then past all conduits leading from previously emptied bag chambers, before the output port is encountered. In this manner, residual fluid output from each bag chamber is pushed through the manifold and out through the output port by fluid from subsequently emptied bag chambers.

In order for the fluid flow to be further regulated (e.g., to prevent unintentional fluid flow from the bag through to the output port), it is desirable that the check valves be controllable as to when flow is permitted therethrough. This can be accomplished in a number of ways, depending on the type of check valve employed. For example, a valve can be employed having a threshold operating pressure (i.e., a cracking pressure) that opens the valve. The cracking pressure of the valve may be any pressure suitable for the intended application. Suitable cracking pressures should be no higher than the pressure generated by the pump apparatus, yet high enough to prevent unintentional flow through the manifold. Preferred cracking pressures can be in the range of about 0.25 lbs per square inch up to about 2 lbs per square inch. It is more preferred that the cracking pressures be in the range of about 0.50 lbs per square inch up to about 1 lbs per square inch. In a most preferred embodiment, the cracking pressure is about 0.75 lbs per square inch. The cracking pressures should be consistent in a given direction of fluid flow. Thus, the check valves associated with the chamber ports and the output port can have one cracking pressure while the check valve(s) associated with the bulk fill port has a different cracking pressure. Due to economies of scale, it presently preferred that the valve types and cracking pressures be consistent throughout the manifold.

An administration set is optionally provided in one embodiment of the present invention. The administration set comprises a length of medical grade tubing, such as a micro-bore tube, or the like, with structures at each end: at one end (proximal end) for connecting the tubing to the output port of the manifold and at the opposite (distal) end for connection to a standard intravenous-type needle. Standard luer connectors, needleless connectors, or the like may be used in the practice of the present invention.

The administration set may be further configured to regulate the rate of fluid administration to the patient. It is necessary to know the pressure generated by the pump/manifold combination in order to calibrate the delivery rate of the administration set. The devices of the present invention may be configured so that the pump apparatus generates predictable fluid pressures based on the volume of solution in each chamber. Using the predictable fluid pressures, the flow rate from the integral container may be selectable using administration sets having predetermined tubing lengths and inner diameters. The flow rate through the administration set is selected by varying the microbore tubing's inner diameter and length. The relationship is approximated by Poiseulle's equation:

$$Q = \frac{Δ p \cdot π \cdot {D}^{4}}{128 \cdot μ \cdot L} \qquad Equation 1$$

Where Q is the flow rate, Δp is the pressure drop across a flow controlling orifice, D is the inside diameter of the orifice, μ is the dynamic viscosity of the fluid and L is the length of the orifice. Thus, any structures included in the administration set will effect the flow rate in a predictable and calculable manner. Structures contemplated for optional incorporation into the administration set include particulate filters, air elimination filters, fluid flow restrictors, flow indicators, drop counters, drip chambers, pressure indicators, and the like. The administration set may further comprise a clamp, or the like, for stopping fluid flow, as desired.

In another embodiment of the present invention there is provided a restrictor set for attachment to the distal end of the administration set. In this manner, the rate of fluid flow can be altered with the simple addition of a restrictor set, rather than by re-engineering the administration set. Of course, the maximum fluid flow rate will be determined by the configuration of the administration set, with fine-tuning to slower rates provided by the restrictor set. Restrictor sets may be located at a variety of positions in a flow path, such as in a chamber, in a conduit, in a manifold, etc.

The integral containers of the present invention may be provided to a user (e.g., a physician or pharmacist) in an unfilled state for subsequent filling with fluids deemed appropriate by the user; that is, the bags may be configured by a clinician or pharmacist to deliver a regimen of fluids deemed advantageous to a particular patient. Alternatively, one or more, and preferably all, chambers within the integral container may be provided to the user pre-filled with fluids to be delivered in a predetermined sequence.

The integral containers and methods described herein can provide a methodology by which a course of therapy involving multiple fluids can be preconfigured and stored, e.g., in a hospital or pharmacy, for “off the shelf” delivery to the clinician or patient. Additionally, the integral containers and methods described herein allow for the careful preselection of fluids, to ensure that none of the fluids to be delivered to a patient from the integral container will interact adversely with other fluids to be delivered from the same integral container. The present invention contemplates that any compounds or groups of compounds that may be delivered in a fluid format may be delivered to a patient in accordance with the foregoing description. An exemplary list of suitable compounds is provided below.

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

antimigraine agents (e.g., ergotamine tartrate, propanolol hydrochloride, isometheptene mucate, dichloralphenazone, and the like);

sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and the like);

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);

antimanic agents (e.g., lithium carbonate),

antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);

antigout agents (e.g., colchicine, allopurinol, and the like);

anticoagulants (e.g., heparin, heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline);

antiplatelet agents (e.g., aspirin, empirin, ascriptin, and the like);

anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

antiparkinson agents (e.g., ethosuximide, and the like);

antidepressants (e.g., doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);

antihypertensive agents (e.g., trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine, and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antifungal agents (e.g., griseofulvin, keloconazole, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);

anti-infectives (e.g., GM-CSF);

bronchodialators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), and the like;

hypoglycemic agents (e.g., human insulin, purified beef insulin, purified pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, and the like);

nucleic acids (e.g., sense or anti-sense nucleic acids encoding any protein suitable for delivery by inhalation, including the proteins described herein, and the like);

agents useful for erythropoiesis stimulation (e.g., erythropoietin);

antiulcer/antireflux agents (e.g., famotidine, cimetidine, ranitidine hydrochloride, and the like); and

antinauseants/antiemetics (e.g., meclizine hydrochloride, nabilone, prochlorperazine, dimenhydrinate, promethazine hydrochloride, thiethylperazine, scopolamine, and the like).

This list is not intended to be limiting. Additional agents contemplated for delivery employing the devices and methods described herein include agents useful for the treatment of diabetes (e.g., activin, glucagon, insulin, somatostatin, proinsulin, amylin, and the like), carcinomas (e.g., taxol, interleukin-1, interleukin-2 (especially useful for treatment of renal carcinoma), and the like, as well as leuprolide acetate, LHRH analogs (such as nafarelin acetate), and the like, which are especially useful for the treatment of prostatic carcinoma), endometriosis (e.g., LHRH analogs), uterine contraction (e.g., oxytocin), diuresis (e.g., vasopressin), cystic fibrosis (e.g., Dnase (i.e., deoxyribonuclease), SLPI, and the like), neutropenia (e.g., GCSF), lung cancer (e.g., beta 1-interferon), respiratory disorders (e.g., superoxide dismutase), RDS (e.g., surfactants, optionally including apoproteins), and the like.

Presently preferred indications which can be treated employing the device and methods described herein include diabetes, carcinomas (e.g., prostatic carcinomas), bone disease (via calcium regulation), cystic fibrosis and breathing disorders (employing bronchodilators), and the like.

While the invention has been described in detail with reference to certain preferred embodiments thereof, it will be understood that modifications and variations are within the spirit and scope of that which is described and claimed.

What is claimed is:

1. A method of providing a therapy regimen to a patient, said therapy regimen comprising the delivery in a predetermined sequence of a plurality of fluids from an integral container to said patient, said integral container comprising said plurality of fluids contained within separate non-fluidly connected chambers, the method comprising:
delivering said plurality of fluids from said separate non-fluidly connected chambers to said patient in said predetermined sequence.

2. The method of claim 1, wherein said plurality of fluids are delivered in a predetermined sequence by exerting positive pressure on said separate non-fluidly connected chambers, whereby said fluids are expressed from said chambers in said predetermined sequence.

3. The method of claim 2, wherein said positive pressure is created by compression of said separate non-fluidly connected chambers in a predetermined sequence.

4. The method of claim 1, wherein said plurality of fluids are delivered in a predetermined sequence by exerting negative pressure on said separate non-fluidly connected chambers, whereby said fluids are extracted from said chambers in said predetermined sequence.

5. The method of claim 4, wherein said negative pressure is created by pumping said fluids from said separate non-fluidly connected chambers in a predetermined sequence.

6. The method of claim 5, wherein said plurality of fluids flow from said separate non-fluidly connected chambers to a manifold comprising a separate input port in fluid communication with each said separate non-fluidly connected chamber and at least one common port, whereby said fluids flow through their respective input port to said common port in said predetermined sequence, and wherein said negative pressure is created downstream from said common port.

7. The method of claim 5, wherein negative pressure is exerted on each said separate non-fluidly connected chamber by a separate pump.

8. The method of claim 7, wherein each said separate pump is controlled by a programmable interface.

9. The method of claim 1, wherein said plurality of fluids are delivered in a predetermined sequence by gravity feed from said separate non-fluidly connected chambers, whereby said fluids flow from said chambers in said predetermined sequence.

10. The method of claim 9, wherein said plurality of fluids are delivered in a predetermined sequence by a differential hydrostatic head height in two or more separate non-fluidly connected chambers.

11. The method of claim 1, wherein said plurality of fluids flow from said separate non-fluidly connected chambers to a manifold comprising a separate input port in fluid communication with each said separate non-fluidly connected chamber and at least one common port, whereby said fluids flow to said common port in said predetermined sequence.

12. The method of claim 1, wherein flow from one or more of said separate non-fluidly connected chambers is controlled by valves.

13. The method of claim 12, wherein said valve(s) are independently selected from the group consisting of umbrella valves, disc valves, poppet valves, duckbill valves, ball valves, flapper valves, shuttle valves, gate valves, slit membranes, and check valves.

14. The method of claim 1, wherein flow from one or more of said separate non-fluidly connected chambers is controlled by an active control device.

15. The method of claim 14, wherein said active control device is selected from the group consisting of a stopcock, a pinch clamp, a pneumatically controlled valve, a vacuum controlled valve, a mechanically controlled valve, a hydraulically controlled valve, and an electrically controlled valve.

16. The method of claim 1, wherein flow from one or more of said separate non-fluidly connected chambers is controlled by a passive control device.

17. The method of claim 1, wherein two or more chambers in said integral container become fluidly connected prior to or during delivery of said plurality of fluids, whereby a single chamber is formed.

18. A fluid delivery device, comprising:
an integral container comprising a plurality of fluids contained within separate non-fluidly connected chambers;
wherein said fluid delivery device is configured and arranged to deliver said plurality of fluids from said separate non-fluidly connected chambers to said at least one common port in a predetermined sequence.

19. The fluid delivery device of claim 18, further comprising a manifold comprising a separate input port in fluid communication with each said separate non-fluidly connected chamber and at least one common port.

20. The fluid delivery device of claim 18, further comprising one or more pumping elements.

21. The fluid delivery device of claim 20, wherein said one or more pumping elements exert positive pressure on said separate non-fluidly connected chambers.

22. The fluid delivery device of claim 21, wherein said positive pressure compresses said separate non-fluidly connected chambers in a predetermined sequence.

23. The fluid delivery device of claim 20, wherein said one or more pumping elements exert negative pressure on said separate non-fluidly connected chambers.

24. The fluid delivery device of claim 23, wherein said negative pressure pumps said fluids from said separate non-fluidly connected chambers in a predetermined sequence.

25. The fluid delivery device of claim 24, wherein said one or more pumping elements are downstream from said manifold common port.

26. The fluid delivery device of claim 24, wherein negative pressure is exerted on each said separate non-fluidly connected chamber by a separate pumping element.

27. The fluid delivery device of claim 18, wherein two or more separate non-fluidly connected chambers comprise different hydrostatic head heights.

28. The fluid delivery device of claim 18, wherein flow from one or more of said separate non-fluidly connected chambers is controlled by valves.

29. The fluid delivery device of claim 28, wherein said valve(s) are independently selected from the group consisting of umbrella valves, disc valves, poppet valves, duckbill valves, ball valves, flapper valves, shuttle valves, gate valves, slit membranes, and check valves.

30. The fluid delivery device of claim 18, wherein flow from one or more of said separate non-fluidly connected chambers is controlled by an active control device.

31. The fluid delivery device of claim 30, wherein said active control device is selected from the group consisting of a stopcock, a pinch clamp, a pneumatically controlled valve, a vacuum controlled valve, a mechanically controlled valve, a hydraulically controlled valve, and an electrically controlled valve.

32. The fluid delivery device of claim 18, wherein flow from one or more of said separate non-fluidly connected chambers is controlled by a passive control device.

33. The fluid delivery device of claim 18, wherein two or more chambers in said integral container become fluidly connected prior to or during delivery of said plurality of fluids, whereby a single chamber is formed.

34. The fluid delivery device of claim 20, wherein said one or more pumping elements are controlled by a programmable interface.
